(19) United States
(12) Patent Application Publication
(10) Pub. No.: US 2025/0347599 A1
(43) Pub. Date: Nov. 13, 2025

(54) Modular Material Testing Apparatus for Material Testing of a Specimen

(71) Applicant: 4a Engineering GmbH, Traboch (AT)

(72) Inventors: Martin SCHWAB, Graz (AT); Reinhard HAFELLNER, Spielberg (AT); Stefan RIEMELMOSER, Mautern in Steiermark (AT); Bernhard JILKA, Zellerndorf (AT); Harald SCHMID, Leoben (AT)

(21) Appl. No.: 18/878,724

(22) PCT Filed: Jun. 27, 2023
(86) PCT No.: PCT/EP2023/067527

§ 371 Date: Dec. 24, 2024

(30) Foreign Application Priority Data
Jun. 27, 2022 (DE) 102022115925.6

(51) Int. Cl. G01N 3/04; G01N 33/00

(52) U.S. Cl. CPC G01N 3/04; G01N 33/0078; G01N 2203/0405

(57) ABSTRACT

An apparatus for material testing of a specimen. The apparatus-includes a specimen holder for holding a specimen to be tested, a rod arrangement for moving in direction to the specimen holder for transmitting a mechanical load to the specimen, an actuator for moving at least one of the rod arrangement and the specimen holder with respect to each other along a horizontal impact direction, and a supporting base, onto which the specimen holder, the rod arrangement and the actuator are mounted. A specimen holder arrangement is detachably coupled to a holder accommodation section of the supporting base.

### CROSS REFERENCE TO RELATED APPLICATIONS

This application is a national stage application, filed under 35 U.S.C. § 371, of International Patent Application No. PCT/EP2023/067527, filed on Jun. 27, 2023, claiming priority of patent application Ser. No. 10/202,2115925.6 filed on Jun. 27, 2022, in Germany, the disclosures of these patent applications being incorporated by reference herein in their entirety.

### TECHNICAL FIELD

The present disclosure relates to an apparatus for material testing of the specimen, in particular a battery, and a method for material testing of the specimen.

### TECHNOLOGICAL BACKGROUND

In material testing, it is necessary to expose material samples to a variety of different types of stress. For example, the material sample is subjected to tensile or compressive stress over a certain period of time. For example, a testing machine may be designed to provide a tensile testing, wherein the material sample is stretched by a specific tensile force. Furthermore, impact tests and bending flexural tests may be conducted by other test machines, wherein an impact element is pressed against the material sample for testing purposes. In addition, testing machines are usually designed for different types of loading speed, e.g. universal testing machines can be used for quasi-static testing only (low speeds), servo-hydraulic machines are usually used for cyclic fatigue testing and special purpose machines (special servo-hydraulic or split Hopkinson bar) for high-speed loading conditions.

The forces can be applied statically or dynamically. Since particularly high forces are at work during testing, the demands on the testing machines are very high, especially with regard to durability and vibration safety. For this reason, testing machines are usually only designed for one load case in order to provide sufficient testing performance for this one load case.

Additionally, the fixation and the alignment of the specimen to be tested has to be very exact and robust, so that the change over time between the test of two specimen takes long setup time of the test arrangement.

### SUMMARY

Hence, there may be a need to provide an apparatus for material testing of a plurality of specimen in an efficient manner.

This need may be met by an apparatus and a method for material testing of a specimen as described by the subject matters of independent claims.

According to a first aspect an apparatus for material testing of a specimen is described. The apparatus comprises a specimen holder for holding a specimen to be tested, a rod arrangement for moving in direction to the specimen holder for transmitting a mechanical load to the specimen, an actuator for moving at least one of the rod arrangement and the specimen holder with respect to each other along a horizontal impact direction, and a supporting base, onto which the specimen holder, the rod arrangement and the actuator are mounted, wherein the specimen holder is detachably coupled to a holder accommodation section of the supporting base.

According to further aspect, a method for material testing of a specimen by an above-described apparatus is presented. According to the method, the specimen holder is detachably coupled to the holder accommodation section of the supporting base and at least one of the rod arrangement and the specimen holder arrangement is moved with respect to each other for transmitting a mechanical load to the specimen for material testing the specimen.

### OVERVIEW OF EMBODIMENTS

The specimen to be tested by the above-described apparatus may be a material part, such as a metal or plastic element, which may have a sheet-like shape or a solid body shape. Furthermore, the specimen to be tested may be a partial product, such as a semifinished or finished device. For example, the specimen to be tested may be a battery cell, an arrangement of several cells, a battery module or a battery pack. The device may be used to test material mechanical properties such as tensile testing and impact testing battery puncture tests are also possible. Hence, the specimen holder may hold the device for example in such a manner, that the rod arrangement is adapted for transmitting a mechanical load to for example housing of the device, in order to provide a respective material test of housing.

The specimen holder arrangement comprises for example a specimen holder designed for holding the specimen specifically in a detachable manner. For example, the specimen holder may comprise clamping elements for clamping the specimen to be tested. Furthermore, the specimen holder may comprise a magnet, in particular a permanent magnet or an electromagnetic magnet, in order to fix the specimen, in particular a metallic specimen, detachably to the holding device. Furthermore, the specimen holder may comprise a chamber, in which the specimen is arranged and into which the rod arrangement may movably enter. Through an opening of the chamber, an impact element of the rod arrangement reaches the sample attached to the sample holder.

The rod arrangement comprises an impact section, which is designed for being pressed against the specimen to be tested. Thereby, the rod arrangement is configured for moving in the direction to the specimen holder. In a further exemplary embodiment, the rod arrangement is configured for transmitting a tractive force to the specimen. The rod arrangement is driven by the actuator and can be moved at an adjustable speed and with an adjustable impact force or tractive force to the specimen. The rod arrangement may comprise an impact element and a force transmitting element, in particular a force transmitting rod, coupled to the actuator. The impact element is in particular harder than the specimen to be tested. Furthermore, the impact element may comprise a conical shape or a pyramid having an impact tip. The impact element may also comprise a hemispherical shape having a round and ball shape impact section. The impact element may also comprise an impact edge having a longitudinal extension or e.g. an impact spike/pin for applying a punctual force. Hence, the press/rod arrangement is configured for transmitting a mechanical load to the specimen. Depending on the actual specimen holder, a variety of mechanical loads can be transmitted, such as e.g. compressive loads, tensile loads, shear loads and/or bending loads. For example, the rod arrangement may load the specimen, for example with high frequency for performing cyclic or fatigue testing, or (with the same arrangement) the rod arrangement may load the specimen statically. Also, the rod arrangement may load the specimen at high speeds for performing high strain rate testing. By the rod arrangement controlled by the actuator a force during a static test may be generated inconstantly. For example, in a static test, a very slow movement of the rod arrangement with continuously increasing force up to a specified level or until rupture of the specimen is provided. However, also a so-called creep test is possible, wherein the force is kept constant over a longer period of time. During a dynamic test, a faster movement of the rod arrangement with e.g. a pre-acceleration phase may be provided, so that the specimen or load introduction device of the specimen holder is hit at a specified speed and/or impact energy. All of these test cases can be performed by the described material testing apparatus.

For example, during an above-described static creep test, the apparatus provides a constant force over a longer period of time. However, the force may not be constant. In a static test, very slow movement of the rod arrangement with a continuously increasing force up to a specified level or until rupture of the specimen may be provided. In a dynamic test, a faster movement of the rod arrangement with a pre-acceleration phase so that the specimen or load introduction device of the specimen holder is hit with a specified velocity and/or impact energy.

The supporting base is designed for supporting the specimen holder arrangement, the rod arrangement and the actuator. The specimen holder arrangement, the rod arrangement and the actuator are mounted (directly or indirectly via coupling supporting elements) to the supporting base. The supporting base is formed robustly, for example by a framework of steel rods which can be arranged on the ground. The supporting base transfers respective weight forces and dynamic forces to the ground.

The actuator may be for example an electro motor or servo motor for driving at least one of the rod arrangement and the specimen holder arrangement in a desired speed along the impact direction and with a desired impact force. Specifically, a constant speed or an inconstant speed, i.e. an acceleration or deceleration, within the range of 0 m/s to 12 m/s may be adjusted. The electro motor is configured to move the rod arrangement relatively to the specimen holder arrangement along a longitudinal impact direction with a speed to at least 4 m/s, in particular to 10 m/s, further in particular to 12 m/s. A speed of 4 m/s means, that between the rod arrangement and the specimen holder arrangement, a speed between 0 m/s to 4 m/s for transmitting a mechanical load to the specimen can be adjusted. In an exemplary embodiment, a constant speed or an inconstant speed, i.e. an acceleration or deceleration, within the range of 0 m/s to 6 m/s or within the range of 0 m/s to 4 m/s may be adjusted. For example, the rod arrangement may apply a force of 25 kN when being driven with 3.6 m/s against the specimen. The electro motor is configured to adjust the (e.g. constant, inconstant (i.e. acceleration or deceleration)) speed to any speed between 0 m/s to 12 m/s between the rod arrangement and the specimen holder arrangement for transmitting a mechanical load to the specimen. At least one of the rod arrangement and the specimen holder arrangement means that the rod arrangement may be driven to the specimen holder arrangement, the specimen holder arrangement may be driven to the rod arrangement or both, the rod arrangement and the specimen holder arrangement may be both driven and hence moved with respect to each other. Specifically, a constant speed or an inconstant speed, i.e. an acceleration or deceleration, within the range of 0 m/s to 12 m/s may be adjusted. In other words, during a test time interval, the speed may be varied over time.

In an exemplary embodiment, the actuator may also function with pneumatic or hydraulic driving means. In an exemplary, the actuator may be a linear motor. The actuator is in particular configured for providing an impact energy against the specimen of more than 100 J (Joule), in particular more than 200 J, further in particular more than 500 J.

Hence, by the approach of the present disclosure, the specimen holder arrangement comprising for example the specimen holder and the accommodation plate, is detachably coupled to the holder accommodation section of the supporting base. Hence, it is possible to preassemble one or a plurality of specimen holder arrangements with the specimen to be tested before mounting the specimen holder arrangement to the apparatus. Hence, the conduction of the tests with the apparatus and the mounting of the specimen to the specimen holder of the specimen holder arrangement may run in parallel such that a more efficient testing procedure can be provided. Furthermore, as described in an exemplary embodiment below, the replacement of a specimen holder arrangement to the holder accommodation section may be handled self-selecting and automatically by the handling device, such as a robot arm, for example. Hence, a more efficient automatic testing procedure for material testing can be provided.

According to a further exemplary embodiment, the actuator and the rod arrangement are configured for conducting a static test for providing a constant pressing force to the specimen to be tested and/or for conducting a dynamic test for varying the impact force in a predefined time span. In a static test, no movement or a very slow movement of the rod arrangement with continuously increasing force up to a specified level or until rupture of the specimen may be provided. In a dynamic test, a faster movement of the rod arrangement with a pre-acceleration phase so that specimen or load introduction device of the specimen holder is hit with a specified velocity and/or impact energy. Accordingly, also test cycles between static and dynamic load against the specimen can be provided by the rod arrangement.

According to further exemplary embodiment, the impact direction is parallel to a horizontal direction, when the apparatus is arranged on a ground. In other words, the impact direction and hence, the movement direction of the rod arrangement is perpendicular with respect to the gravitational force direction. By applying such a horizontal alignment of the rod arrangement only a minor effect or disturbance by gravity along the impact direction is caused, so that the same undisturbed movement or acceleration in both directions is possible. This approach contradicts many conventional approaches, wherein the impact direction is vertical in order to use the weight of the impact tools to generate a higher impact force.

According to a further exemplary embodiment, the accommodation section comprises a holder accommodation plate onto which the specimen holder is detachably coupled. For example, the accommodation plate may be rigidly fixed to the supporting base. The accommodation plate may comprise for example detachable fixing means, such as screw connections, for fixing the specimen holder detachably to the accommodation plate. The holder accommodation plate may also be rigidly fixed to the specimen holder arrangement such that the accommodation plate and the specimen holder arrangement forms a modular unit which can be easily exchanged.

According to a further exemplary embodiment, the holder accommodation plate comprises at least one mounting pin being engageable in a respective accommodation hole of a coupling plate of the accommodation section. Alternatively, the coupling plate comprises at least one mounting pin being engageable in a respective accommodation hole of a holder accommodation plate of the accommodation section.

Hence, the holder accommodation plate together with the specimen holder arrangement may be moved along a vertical direction onto the coupling plate for engaging the mounting pins in the accommodation holes.

According to a further exemplary embodiment the accommodation section comprises controllable clamping means for fixing the mounting pins into the respective accommodation hole of the coupling plate. In the accommodation hole, a controllable fixing mechanism may be installed. For example, the fixing mechanism may comprise respective clamping means which may be moved in a clamping position, if the mounting pin is arranged within the accommodation hole. The clamping means may be for example movable clamping pins or balls that may be pretensioned in direction of the center of the hole or may be actively driven, e.g. by an electric, pneumatic or hydraulic actuator. Furthermore, a magnetic fixing mechanism may be provided. For example, the mounting pins may be formed of a magnetic (e.g. ferromagnetic) material and inside the accommodation hole, respective electromagnetic elements are provided. Hence, by activating the electromagnetic elements in an accommodation hole, a respective magnetic mounting pin can be fixed.

According to a further exemplary embodiment the apparatus further comprises a lifting mechanism coupled to the coupling plate, wherein the lifting mechanism is configured for lifting the holder accommodation plate from the coupling plate to thereby decoupling the holder accommodation plate from the coupling plate. Hence, if the holder accommodation plate is decoupled from the coupling plate, a respective handling device, such as a robot arm, a conveyor and/or a forklift may be moved between the coupling plate and the holder accommodation plate for moving (automatically) the holder accommodation plate to or away from the apparatus. The lifting mechanism lifts the holder accommodation plate in particular along the vertical direction. However, the lifting mechanism may also be configured as a pushing or pulling mechanism to push or pull the holder accommodation plate along a side direction (horizontal direction), in particular perpendicular to the impact direction, onto or from the coupling plate. For example, the lifting mechanism may push the holder accommodation plate along a side direction onto a conveyor belt arranged adjacent to the coupling plate of the apparatus.

According to a further exemplary embodiment the lifting mechanism comprises at least two, in particular three, lifting pistons configured for being extendable (and retractable) between the holder accommodation plate and the coupling plate for lifting and lowering the holder accommodation plate from the coupling plate. The lifting pistons may be driven by pneumatic, hydraulic or electric actuators.

According to a further exemplary embodiment, the holder accommodation plate comprises at least one accommodation groove, wherein the specimen holder comprises at least one accommodation protrusion. The accommodation protrusion is configured for being slidable within the accommodation groove for detachably coupling the specimen holder to the holder accommodation section. The accommodation groove may be formed in an exemplary embodiment in particular perpendicular to the impact direction.

The holder accommodation section may comprise for example a plurality of accommodation grooves that extend within the horizontal plane and perpendicular to the impact direction. The specimen holder may comprise respective accommodation protrusions that may be slid in the grooves along a direction perpendicular to the impact direction. The accommodation protrusions may form for example protruding edges or ridges that extend along a straight longitudinal direction. The accommodation protrusions may form an exemplary embodiment also protruding studs or pins being insertable in the accommodation groove.

The accommodation groove may be formed in an exemplary embodiment in particular perpendicular to the impact direction. Hence, the forces induced by the impact of the rod arrangement at the specimen to be tested are directed perpendicular to the sliding direction of the accommodation protrusion within the grooves such that the force can be transferred directly from the specimen holder into the accommodation section. Hence, a robust and simple detachable fixation of the specimen holder to the accommodation section of the supporting base is provided.

According to a further exemplary embodiment, the accommodation section comprises a coupling plate configured for detachably coupling the accommodation plate. Hence, the accommodation plate together with specimen holder arrangement, for example the specimen holder of the specimen holder arrangement, forms a modular unit which can be easily exchanged and may be preassembled before being mounted to the coupling plate. The coupling plate may be rigidly fixed to the supporting base. Additionally, the accommodation plate and the coupling plate form a surface contact instead of point contact, such that an improved and stable alignment and fixation between the coupling plate and the accommodation plate is provided. According to this exemplary embodiment, a plurality of specimen holder arrangements each mounted on a respective accommodation plate (rigidly or detachably) may be preassembled with a respective specimen outside of the test apparatus. As described below, by a manipulator (e.g. robot arm or a conveyor), the accommodation plate together with the specimen holder arrangement can be transferred to or away from the test apparatus automatically. Hence, an automated process for testing a plurality of specimens under e.g. different test conditions can be provided.

In another exemplary embodiment, the accommodation plate forms the accommodation protrusions, and the specimen holder may form the accommodation grooves.

According to a further exemplary embodiment, the accommodation plate comprises at least one coupling groove and the coupling plate comprises a coupling protrusion, wherein the coupling protrusion and the coupling groove are formed in such a manner that the coupling protrusion is slidable within the coupling groove.

The coupling protrusions may form for example protruding edges or ridges that extend along a straight longitudinal direction. The coupling protrusions may form in an exemplary embodiment also protruding studs or pins being insertable in the coupling groove.

According to an exemplary embodiment, the coupling groove extends perpendicular to the impact direction. Hence, the forces induced by the impact of the rod arrangement at the specimen to be tested are directed perpendicular to the sliding direction of the coupling protrusion within the grooves such that the force can be transferred directly from the specimen holder into the accommodation plate and further into the coupling plate. Hence, a robust and simple detachable fixation of the accommodation plate to the coupling plate is provided.

In another exemplary embodiment, the accommodation plate on the coupling protrusions and the coupling plate may form the coupling grooves.

According to an exemplary embodiment, the coupling groove comprises tapered sidewalls, in particular with a trapezoidal cross-section. According to an exemplary embodiment, the coupling protrusion is a longitudinal ridge with tapered sidewalls, in particular with a trapezoidal cross-section. Hence, by providing the tapered sidewalls of the coupling groove and/or the coupling protrusion, a self-aligning effect is generated if the accommodation plate is moved towards the coupling plate, in particular along a vertical direction. By providing the tapered sidewalls, the opening of the coupling groove is larger than the width of the coupling groove at the bottom of the coupling groove. Similarly, the free end and top section of tapered coupling protrusion has a smaller width than the bottom section of the coupling protrusion at the location, the coupling protrusion is fixed to the coupling plate. The angle of the tapered sidewall of the coupling groove and the coupling protrusion are similar to provide a proper alignment. Hence, the walls of the coupling groove and the coupling protrusion form a full surface contact instead of a point contact.

According to a further exemplary embodiment, the accommodation section comprises a clamping arrangement configured for selectively holding and moving the accommodation plate to the coupling plate along a clamping direction such that accommodation plate is clampable to the coupling plate. In an exemplary embodiment, the clamping direction is in particular a vertical clamping direction. The clamping direction is the direction along which the accommodation plate is movable to the clamping plate. Hence, the respective clamping force along the clamping direction presses and hence clamps the accommodation plate to the clamping plate. Specifically, when pressing the clamping plate to the accommodation plate, a fixation along the clamping direction is provided. Additionally, due to the above-described clamping protrusions and clamping grooves, a respective fixation perpendicular to the clamping direction, in particular along the impact direction is provided. If the accommodation plate is clamped to the clamping plate, a relative movement between the respective plates is impossible due to the clamping force. However, upon releasing the clamping connection between the accommodation plate and the coupling plate, a detaching of the accommodation plate with respect to the clamping plate in particular perpendicular to the clamping direction and the impact direction is possible. For example, the accommodation plate may slide along the clamping protrusion if no clamping force is generated. Hence, the clamping protrusion and the respective clamping groove of the clamping plate and the accommodation plate helps to adapt and orientate the accommodation plate with respect to the rod arrangement and the impact direction, respectively, whereas the clamping arrangement pressing and clamping the accommodation plate in the direction to the clamping plate helps to fix the accommodation plate rigidly to the coupling plate.

Thus, an efficient process for aligning and fixing the accommodation plate with respect to the rod arrangement is provided, since in the first step the accommodation plate is orientated and aligned with respect to the coupling plate in a self-acting manner by providing the clamping groove/protrusion and in the second step a rigid fixation due to the clamping arrangement is provided.

According to a further exemplary embodiment, the clamping arrangement comprises at least one clamping rod configured for pulling the accommodation plate to the coupling plate along the clamping direction. The clamping rod forms a tension rod that may press the accommodation plate to the clamping plate. Specifically, a plurality of clamping rods may be provided. The clamping rods may be rigidly or detachably fixed to the accommodation plate. Alternatively, the clamping rods may also be rigidly or detachably fixed to the coupling plate. In an exemplary embodiment described below, the clamping rods are detachably fixed to the accommodation plate and may pass the clamping plate.

According to a further exemplary embodiment, the clamping rod is coupled to the accommodation plate and to the coupling plate in such a way that along the clamping direction the coupling rod is fixed to the accommodation plate and slidable with respect to the coupling plate. Hence, the clamping rods are detachably fixed to the accommodation plate and may pass the clamping plate through respective holes in the clamping plate.

According to a further exemplary embodiment, the clamping arrangement comprises a driving system for moving the clamping rod along the clamping direction. The driving system is generated for moving the clamping rod along the clamping direction by means of a driving force generated for example by a respective motor, such as the spindle motor described below. Alternatively, the clamping rods may be formed of threaded bars that may be guided through the coupling plate in may be fixed by a respective lock screw. By screwing the lock screw, the respective clamping force can be generated.

According to a further exemplary embodiment, the driving system comprises a driving plate to which the clamping rod is non movably fixed at least along the clamping direction, wherein the driving plate is movably with respect to the clamping plate. Hence, by moving the driving plate in a longitudinal direction along the clamping direction, the clamping rods fixed to the driving plate are moved as well. For example, a plurality of clamping rods can be fixed to the driving plate, so that only the one driving plate is moved, while a plurality of clamping rods is moved. Hence, it is not necessary to couple each clamping rod individually to a respective driving actuator.

According to a further exemplary embodiment, a spindle is fixed to the coupling plate, wherein the driving plate is movable coupled along the spindle. The driving system comprises a spindle drive mounted on the driving plate for generating a driving force along the spindle. The spindle drive comprises for example a rotatable threaded nut, which is rotated by a driving force. The spindle drive rotates the threaded nut, such that a movement along the spindle, which is coupled to the threaded nut, is provided. The spindle may further be guided through a through hole of the driving plate, such that no coupling between the driving plate and the spindle would interfere with the relative movement of the driving plate in the direction of the coupling direction. Hence, the clamping force is guided from the clamping rods via the driving plate to the coupling plate. All equipment that is necessary for providing a movement and the control of the clamping of the accommodation plate to the coupling plate is arranged on the driving plate. More operational space for providing the coupling arrangement for the accommodation plate is given, since the coupling plate is for example free of any actuators.

According to a further exemplary embodiment, the accommodation plate comprises a clamping groove for accommodating the clamping rod in such a manner that the clamping rod is slidable along the clamping groove and is movably fixed with the accommodation plate along the clamping direction. The clamping groove comprises specifically a lateral opening along a side edge of the accommodation plate. Furthermore, the clamping groove is formed in such a manner, that an undercut (in a cross-sectional view) is formed, such as a C-shape or a T-shape. Hence, the clamping groove comprise a T-shaped cross section and the clamping rod comprises a T-shaped rod end configured for fitting into the T-shaped clamping groove.

The rod end of the clamping rod comprises a respective shape that fits into the clamping groove. Hence, the clamping rod may slide from the side edge into the clamping groove, in particular along a horizontal direction, that is perpendicular to the impact direction. If the clamping force is applied to the clamping rod, a form fit between the clamping rod and the clamping groove having an undercut is generated such that the accommodation plate may be pressed and drawn in the direction to the clamping plate via the clamping rod.

According to a further exemplary embodiment, the supporting base comprises in particular a supporting plate, in particular an aluminum plate, to which at least the clamping arrangement, in particular the coupling plate is mounted. The supporting plate forms a unique and robust basis for the rod arrangement, the actuator and the specimen holder arrangement.

According to a further exemplary embodiment the supporting base comprises at least one vertical shear panel extending between the ground on the one side and the specimen holder arrangement, the rod arrangement and the actuator on the other side, wherein the shear panel is in particular a vertical orientated sheet, in particular a metal sheet. The shear panel is in particular a vertical orientated sheet, in particular a metal sheet. By providing the vertical shear panel, forces (specifically shear forces) extending along the vertical direction and also along the impact direction are absorbed and damped by the vertical shear panel. Specifically, for damping the respective forces, a respective sheet-like panel is sufficient such that also a lightweight solution for damping the vertical forces is provided. The shear panel is configured for having eigenfrequencies along the impact direction of more than 300 Hz. The eigenfrequencies can be adjusted by providing a respective thickness of the shear panel and by using an appropriate material, such as metal.

According to a further exemplary embodiment, the arrangement further comprises a handling device, wherein the handling device is configured to handle the accommodation plate, in particular to move the accommodation plate to or away from the coupling plate. The handling device may be for example a robot arm designed for gripping the accommodation plate and moving the accommodation plate along the horizontal and vertical direction to or from the coupling plate. Furthermore, the handling device may comprise a conveyor device, for example a conveyor belt, that moves the accommodation plate along a sliding direction until the coupling plate. Specifically, a sliding direction and hence the conveying direction is defined along the coupling protrusion and the coupling groove, respectively. A sliding direction may be a horizontal direction perpendicular to the impact direction. Hence, from one lateral side, the accommodation plate may be slid over the coupling plate for providing the material test and after the material test is accomplished, the accommodation plate may move along the sliding direction to and/or away from the coupling plate. Hence, an efficient loading of the apparatus for material testing is provided.

By the above-described coupling arrangement, a sliding mechanism for mounting a specimen holder arrangement together with the accommodation plate manually or automatically is provided. The above-described clamping mechanism provides a mechanism for horizontal fixation as well as for vertical clamping fixation. Hence, by the above-described clamping mechanism, a robust connection of the high stiffness of respective supporting structures, e.g. of more than 600 kN/mm can be provided in the movement during testing.

According to a further exemplary embodiment, the apparatus comprises a control unit for controlling the handling device and the actuator for equipping the holder accommodation section with the specimen holder arrangement and for conducting a material test. The control unit may be coupled to the rod arrangement, the actuator, and the specimen holder arrangement for transmitting control signals in order to control the apparatus such that a self-acting activation of the apparatus and a material test, respectively, can be provided. Additionally, the control unit is coupled to the handling device and the driving system in order to automatically clamp and release the accommodation plate onto which the specimen holder arrangement equipped with the specimen to be tested is preassembled. Hence, by the clamping system between the accommodation plate and the coupling plate, an automatic and self-acting loading and unloading of the material test apparatus can be provided.

Furthermore, a control unit may comprise specimen data including for example design and material parameters of the specimen as well as predefined test procedures, such as information about the pressing force, the frequency of the rod arrangement during a dynamic test, for conducting a respective material test. Hence, by the control unit an automatic loading and unloading of the apparatus can be provided as well as an automatic operation of the material test apparatus is provided.

Hence, the material test of the specimen can be conducted under predetermined climate requirements. For example, a material test can be conducted in a cold environment, such as in temperatures below −40° C., or in a hot environment, such as more than 200° C.

According to a further exemplary embodiment the rod arrangement further comprises a force sensor for measuring the impact force between the rod arrangement, and the specimen to be tested, wherein the force sensor is in particular arranged between an impact element and a force transmitting element of the rod arrangement.

The force sensor may be arranged between the impact element and the force transmitting element. Hence, if the force sensor is arranged close to the impact element, a direct measurement and a proper reachability of the force sensor is possible. Specifically, if the force sensor is mounted close to the impact element at a front and no time delay of force signals during dynamic measurements is generated so that a very exact force measurement is provided. Hence, a direct signal is used instead of a delayed signal e.g. from an engine controller of an actuator unit. The force sensor may be detachably mounted to at least one of the impact element and the force transmitting element. Therefore, a different type of force sensor can be applied for different load cases or a defect for sensor may be exchanged. The force sensor may be a piezoelectric sensor. A piezoelectric sensor is a sensor that uses the piezoelectric effect to measure changes in pressure, acceleration, temperature, strain, or force by converting them to an electrical charge. For a force measurement, the piezoelectric sensor may comprise a thin membrane and a massive base, ensuring that an applied pressure specifically loads the elements in one direction and therefore creates respective electric signals indicative of the applied forces. Additionally or alternatively, also a strain gauge sensor, i.e. a DMS sensor, may be used as a force sensor for a measurement of strain of the rod arrangement.

According to a further exemplary embodiment, the arrangement further comprises an optical measuring device, in particular a high-speed camera, for optically measuring the specimen under test, and/or a displacement sensor for measuring a displacement of a part of the specimen under testing conditions, in particular under a treatment of the specimen with the rod arrangement. Hence, deformations of the specimen under test can be made available.

According to a further exemplary embodiment, the coupling device comprises a coupling pin configured for being insertable in a receiving hole of a coupling section of the apparatus, wherein the coupling pin comprises a receiving section, in particular a groove, for receiving a clamping element, in particular radially movable balls, in particular steel balls, of the apparatus.

The coupling pin extends in particular in a force transmitting direction and a moving direction of the force transmitting rod. The clamping element may be a pin or balls being pretensioned in a radial direction (hence to the center axis of the force transmitting rod). Hence, upon movement of the coupling pin to the actuator, the clamping element (e.g. the clamping pin or balls in the receiving hole of the coupling section of the actuator) are pushed radially outwards until the receiving section, i.e. the groove, of the clamping pin reaches the clamping element. In this position, the clamping element is pushed by the pretension force into the receiving section, such that further relative axial movement (along the center axis of the force transmitting section) is prevented.

The clamping elements may be pretensioned by respective springs. In an exemplary embodiment, the clamping elements are pretensioned in such a manner, that the clamping elements are arranged in a closed position (e.g. where the clamping elements contacts each other), wherein by an opening force (e.g. by pressurized air) the clamping elements are moved in an open position (e.g. spaced apart from each other) such that the pins may be arranged between the clamping elements. Upon removing and deactivating the opening force, the clamping elements move in the closed position and thereby clamp the pins between each other. The clamping elements may also be driven electrically, pneumatically or hydraulically for controlling the pretension force. Hence, also a decoupling of the coupling device is provided.

The coupling device may be in other words a mechanical clamping device comprising for example extending pins extending from the force transmitting rod in a direction to the actuator. The actuator may comprise a coupling section having e.g. receiving holes for the pins. In the receiving holes respective clamping elements may be arranged. The pins may be clamped between the clamping elements for providing a coupling between the force transmitting rod and the actuator.

The coupling device may also form a magnetic coupling. For example, the force transmitting rod may comprise magnetic elements and the actuator comprises at the coupling section a controllable electromagnetic device for selectively coupling the force transmitting rod to the actuator.

Furthermore, the coupling device may also form a screw connection between the force transmitting rod and the actuator.

The actuator may comprise a respective coupling mechanism, for detachably coupling the coupling device. For example, the coupling device may comprise respective coupling pins being insertable in respective coupling holes of the actuator, and vice versa. The coupling holes may include respective clamping elements for clamping the coupling pins of the coupling device. Furthermore, also a controllable magnetic coupling may be provided.

The above-described control device may be coupled to the respective sensors and the respective sensor data may be sent to a data acquisition unit, such as a central server unit, for processing the sensor data. Hence, during a test of the specimen, an exact visualization of the behavior of the specimen under test conditions can be given. The sensor data may also be used for simulation tools and other design processes, for example. By processing the sensor data, also an emergency stop can be automatically initiated.

It has to be noted that embodiments of the disclosure have been described with reference to different subject matters. In particular, some embodiments have been described with reference to apparatus type claims whereas other embodiments have been described with reference to method type claims. However, a person skilled in the art will gather from the above and the following description that, unless otherwise notified, in addition to any combination of features belonging to one type of subject matter also any combination between features relating to different subject matters, in particular between features of the apparatus type claims and features of the method type claims is considered as to be disclosed.

### BRIEF DESCRIPTION OF THE DRAWINGS

The aspects defined above, and further aspects of the present disclosure are apparent from the examples of embodiment to be described hereinafter and are explained with reference to the examples of embodiment. The disclosure will be described in more detail hereinafter with reference to examples of embodiment but to which the disclosure is not limited.

FIG. 1 shows a perspective view of an arrangement for material testing of a specimen according to an exemplary embodiment of the present disclosure.

FIG. 2 shows a perspective view of a clamping arrangement according to an exemplary embodiment of the present disclosure.

FIG. 3 shows a schematical view of a clamping arrangement as shown in FIG. 2.

FIG. 4 shows a schematical view of a respective rod arrangement according to an exemplary embodiment of the present disclosure.

FIG. 5 shows an arrangement specifically with its supporting base according to an exemplary embodiment of the present disclosure.

FIG. 6 and FIG. 7 show an exemplary embodiment of an apparatus providing a rod arrangement for applying a tractive force to the specimen.

FIG. 8 shows a schematic view of a coupling plate comprising accommodation holes according to an exemplary embodiment.

FIG. 9 shows a schematic view of holder accommodation plate of the accommodation section with mounting pins according to an exemplary embodiment.

FIG. 10 and FIG. 11 show schematic views of a coupling device according to an exemplary embodiment.

### DETAILED DESCRIPTION OF EXEMPLARY EMBODIMENTS

The illustrations in the drawings are schematically presented. It is noted that in different figures similar or identical elements are provided with the same reference signs.

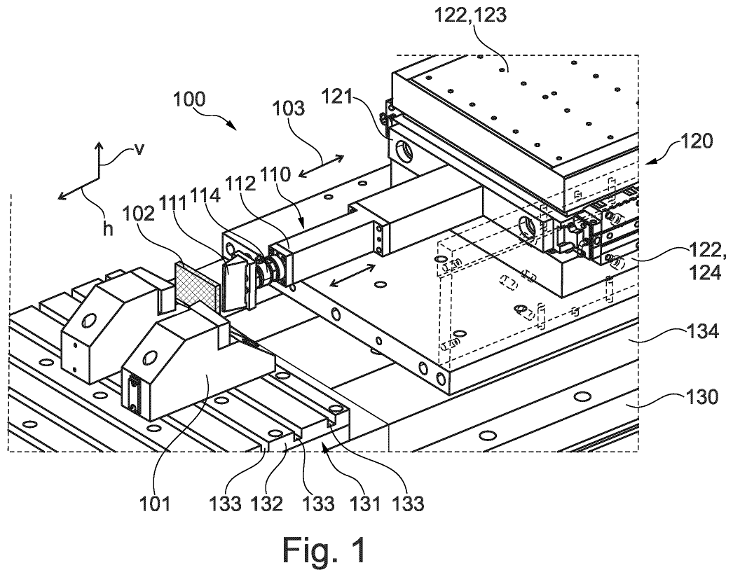

FIG. 1 shows a perspective view of an arrangement 100 for material testing of a specimen 102 according to an exemplary embodiment of the present disclosure. The apparatus 100 comprises a specimen holder arrangement comprising a specimen holder 101 for holding a specimen 102 to be tested, a rod arrangement 110 for moving in direction to the specimen holder 101 for transmitting a mechanical load to the specimen 102, an actuator 120 for moving the rod arrangement 110 relatively to the specimen holder 101 along a horizontal impact direction 103, and a supporting base, onto which the specimen holder 101, the rod arrangement 110 and the actuator 120 are mounted, wherein the specimen holder is detachably coupled to a holder accommodation section 131 of the supporting base.

The specimen holder 101 of the specimen holder arrangement is designed for holding the specimen 102 specifically in a detachable manner to a holder accommodation section 131 of a supporting base 130. For example, the specimen holder 101 may comprise clamping elements for clamping the specimen 102 to be tested. By providing a detachable specimen holder arrangement, the respective specimen holder 101 may be preassembled with the specimen 101 to be tested before fixing the holder 101 to the holder accommodation section 131.

The test apparatus 100 further comprises a supporting plate 134 of the supporting base 130, onto which the specimen holder 101, the rod arrangement 110 and the electromechanical actuator 120 are mounted (directly or indirectly via coupling supporting elements). The supporting base 130 transfers respective weight forces and dynamic forces to the ground.

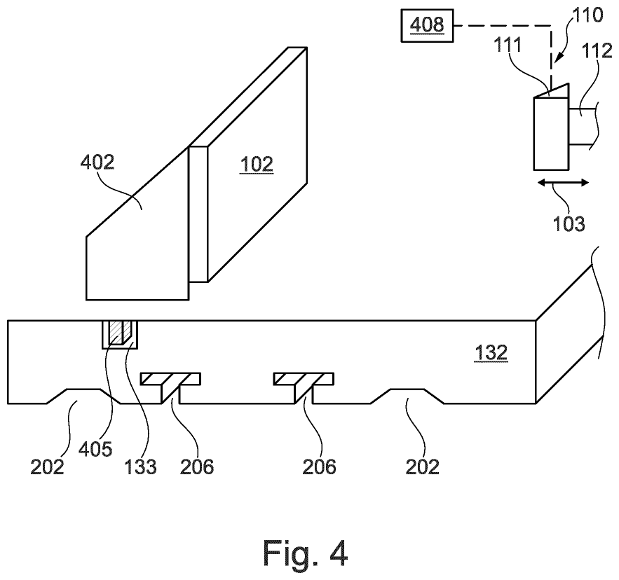

The holder accommodation section 131 comprises at least one accommodation plate 132 with accommodation grooves 133, wherein the specimen holder 101 comprises at least one accommodation pin as accommodation protrusion 405 (see FIG. 4). For example, the accommodation plate 132 may be rigidly fixed to the supporting base 130. The accommodation pin is slidable into the accommodation groove 133 for detachably coupling the specimen holder 101 to the holder accommodation plate 132. The accommodation groove 133 is formed in particular perpendicular to the impact direction 103. The holder accommodation plate 132 may comprise for example a plurality of accommodation grooves 133 that extend within the horizontal plane and perpendicular to the impact direction 103. The respective accommodation pins of the specimen holder 101 slid in the accommodation grooves 133 along a direction perpendicular to the impact direction 103. Hence, the forces induced by the impact of the rod arrangement 110 at the specimen 102 to be tested are directed perpendicular to the sliding direction of the accommodation pins within the accommodation grooves 133 such that the force can be transferred directly from the specimen holder 101 into the accommodation plate 132.

The rod arrangement 110 comprise an impact element 111 having an impact section which is designed for being pressed against the specimen 102 to be tested. Thereby, the rod arrangement 110 is configured for moving in the direction to the specimen holder 101. The rod arrangement 110 is driven by the electromechanical actuator 120 and can be moved in an adjustable speed and an adjustable impact force to the specimen 102.

The actuator 120 may be for example an electro motor or servo motor for driving the rod arrangement 110 in a desired speed along the impact direction 103 and with a desired impact force. In the exemplary embodiment, the actuator 120 is a linear motor. The electromechanical actuator 120 is in particular configured for providing an impact energy against the specimen 102 of more than 100 J.

The actuator 120 is configured to adjust at least one of the rod arrangement 110 and the specimen holder arrangement with respect to each other with a constant speed or an inconstant speed, i.e. an acceleration or deceleration, within the range of 0 m/s to 12 m/s. Therefore, a plurality of load cases can be applied. For example, the rod arrangement 110 may impact the specimen 102 with a high frequency, or the rod arrangement 110 may transfer mechanical load (tension, tractive force or press force) statically against the specimen 102. Hence, by the mentioned apparatus 100, a plurality of different load cases for material testing may be applied within one and the same apparatus.

The apparatus 100 is configured that the impact direction 103 is parallel to a horizontal direction h, when the apparatus 100 is arranged on a ground. In other words, the impact direction 103 and hence, the movement direction of the rod arrangement 110 is perpendicular with respect to the gravitational force direction (along the vertical direction v). By applying such a horizontal alignment of the rod arrangement 110 only a minor effect or disturbance by gravity along the impact direction 103 is caused, so that the same undisturbed movement or acceleration in both directions is possible. Hence, a static test for providing a constant pressing force to the specimen 102 to be tested and/or for conducting a dynamic test for varying the impact force in a predefined time span can be provided. The electromechanical actuator 120 and the rod arrangement 110 are configured for pressing the rod arrangement 110 to the specimen 103 with a pressing force of more than 5 kN.

Furthermore, the actuator 120 is configured to move the rod arrangement 110 along the impact direction 103 to and away from the specimen holder 101. Hence, it is not only possible to provide a pressing force against the specimen 102 in a direction to the specimen 102, but also a pulling force in a direction away from the specimen 102. Hence, tension tests and a variety of load condition between pressing/pending tests and tension tests can be provided by the arrangement. In order to provide the tension tests, the rod arrangement 110 can be rigidly fixed to the specimen.

The rod arrangement 110 comprises the impact element 111 and a force transmitting rod 112, coupled to the actuator 120. The impact element 111 is in particular harder than the specimen 102 to be tested. Furthermore, the impact element 111 may comprise in the shown exemplary embodiment an impact edge having a longitudinal extension.

The force transmitting rod 112 provides the coupling between the impact element 111 and the actuator 120. The force transmitting rod 112 may be coupled to the movable part, e.g. a slide 121 of the electromechanical actuator 120. The slide 121 has a weight of more than 100 kg. Hence, a high impact energy due to a high mass of more than 100 kg is provided.

The impact element 111 is detachably coupled to the force transmitting rod 112. Hence, impact elements 111 of different design and shape may be exchanged in order to test the specimen 102 with different load cases, for example. Furthermore, if an impact element 111 is damaged, a respective change of impact elements 111 is possible.

The rod arrangement 110 further comprises a force sensor 113 for measuring the impact force between the impact element 111 and the specimen 102 to be tested. The force sensor 113 is arranged between the impact element 111 and the force transmitting rod 112. Hence, if the force sensor 113 is arranged close to the impact element 111, a direct measurement and a proper reachability of the force sensor 113 is possible. Specifically, if the force sensor 113 is mounted close to the impact element 111 at a front and no time delay of force signals during dynamic measurements is generated so that a very exact force measurement is provided.

The force sensor 113 is detachably mounted to at least one of the impact element 111 and the force transmitting rod 112. The, the force sensor 113 may be a piezoelectric sensor.

In the exemplary embodiment, the actuator 120 is a linear motor comprising a movable slide 121 to which the rod arrangement 110 is coupled and a stator 122 extending along the impact direction 103. The slide 121 is drivable along the impact direction 103 relatively to the stator 122 by electromechanical driving forces generatable between the stator 122 and the slide 121. The linear motor produces a linear driving force along its length and hence along the impact direction 103. A typical mode of operation is as a Lorentz-type actuator, in which the applied force is linearly proportional to the current and the magnetic field. By the linear motor, the impact force and the speed of the rod arrangement 110 can be adjusted precisely.

The force transmitting rod 112 is coupled to the movable slide 121, wherein the force transmitting rod 112 has in particular a length along the impact direction 103 which is longer than a traveling distance of the slide 121 along the impact direction 103. Hence, the maximum traveling distance of the slide 121 can be used to move the rod arrangement 110, since the impact element 111 arranged on the force transmitting rod 112 is not in conflict with structural elements of the linear motor, because of the length of the force transmitting rod 112.

In the embodiment, the stator 122 is made of a rectangular, stator table 123 and a further stator table 124. The stator tables 123, 124 comprise a length along the impact direction 103 and a width orthogonal to the impact direction 103, wherein the length is longer than the width of the stator tables 123, 124.

The slide 121 is slidably arranged between the stator table 123 and the further stator table 124. Hence, by providing two stator tables 123, 124 which sandwich the slide 121, a stronger magnet field for driving the slide 121 may be provided.

Hence, the specimen holder 101 is detachably coupled to the holder accommodation section 131 of the supporting base 130. Hence, it is possible to preassemble one or a plurality of specimen holders 101 with the specimen 102 to be tested before mounting the specimen holder 101 to the apparatus.

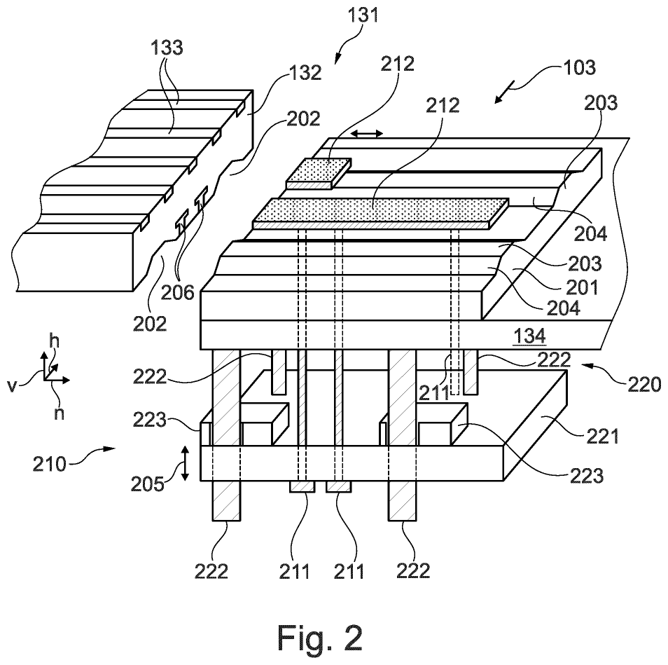

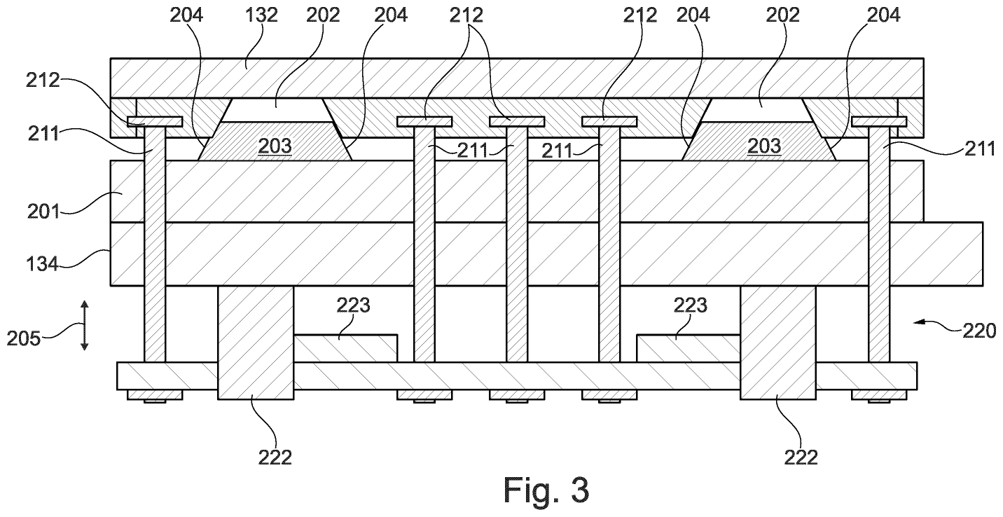

FIG. 2 and FIG. 3 show exemplary embodiments of a clamping arrangement 210 according to an exemplary embodiment of the present disclosure.

The accommodation section 131 comprises a coupling plate 201 configured for detachably coupling the accommodation plate 132. Hence, the accommodation plate 132 together with specimen holder arrangement may be preassembled before being mounted to the coupling plate. 201 The coupling plate 201 may be rigidly fixed to the supporting plate 134. Additionally, the accommodation plate 132 and the coupling plate 201 form a surface contact.

The accommodation plate 132 comprises at least one coupling groove 202 and the coupling plate 201 comprises a coupling protrusion 203, wherein the coupling protrusion 203 and the coupling groove 202 are formed in such a manner that the coupling protrusion 203 is slidable within the coupling groove 202.

The coupling protrusions 203 form protruding edges or ridges that extend along a straight longitudinal direction. The coupling grooves 202 extend perpendicular to the impact direction 103. Hence, the forces induced by the impact of the rod arrangement 120 at the specimen 102 to be tested are directed perpendicular to the sliding direction of the coupling protrusion 203 within the grooves 202 such that the force can be transferred directly from the specimen holder 101 into the accommodation plate 132 and further into the coupling plate 201.

The coupling grooves 202 comprise tapered sidewalls, in particular with a trapezoidal cross-section. Also, the coupling protrusions 203 form longitudinal ridges with tapered sidewalls 204, in particular with a trapezoidal cross-section. Hence, by providing the tapered sidewalls 204 of the coupling groove 202 and/or the coupling protrusion 203, a self-aligning effect is generated if the accommodation plate 132 is moved towards the coupling plate 201, in particular along a vertical direction v. Hence, if pulling the accommodation plate 132 downwards along the coupling direction 205, horizontal and vertical movement is prevented due to locking of the angled surfaces of the trapezoidal blocks.

Specifically, the supporting plate 134 is an aluminum plate, to which at least the clamping arrangement 210, in particular the coupling plate 201 is mounted. In an exemplary embodiment, the supporting plate 134 and the coupling plate 134 may be integrally and monolithically formed of one piece.

The accommodation section 131 further comprises a clamping arrangement 210 for moving the accommodation plate 132 to the coupling plate 201 along a clamping direction 205 such that accommodation plate 132 is clampable to the coupling plate 201. The clamping direction 205 is a vertical clamping direction. The clamping direction 205 is the direction along which the accommodation plate 132 is movable to the clamping plate 201. A respective clamping force acts along the clamping direction 205 and hence clamps the accommodation plate 132 to the clamping plate 201 such that a fixation along the clamping direction is provided. Additionally, due to the above-described tapered clamping protrusions 203 and clamping grooves 202, a respective fixation perpendicular to the clamping direction 205, in particular along the impact direction 103 is provided. If the accommodation plate 132 is clamped to the clamping plate 201, a relative movement between the respective plates 132, 201 is impossible due to the clamping force. However, upon releasing the clamping connection between the accommodation plate 132 and the coupling plate 201, a movement of the accommodation plate 132 with respect to the clamping plate 201 in particular perpendicular to the clamping direction 205 and the impact direction 103 is possible. For example, the accommodation plate 132 may slide along the clamping protrusion 203 if no clamping force is generated. Hence, the clamping protrusion 203 and the respective clamping groove 202 of the clamping plate 201 and the accommodation plate 132 helps to adapt and orientate the accommodation plate 132 with respect to the rod arrangement 120 and the impact direction 103, respectively, whereas the clamping arrangement 210 pressing and clamping the accommodation plate 132 in the direction to the clamping plate 201 helps to fix the accommodation plate 132 rigidly to the coupling plate 132.

For providing the clamping force along the vertical direction v, the clamping arrangement 210 comprises clamping rods 211 configured for pulling the accommodation plate 132 to the coupling plate 201 along the clamping direction 205. The clamping rods 211 are detachably fixed to the accommodation plate 132. The clamping rods 211 pass the clamping plate 201 through respective holes in the clamping plate 201. The clamping rods 211 are coupled to the accommodation plate 132 and to the coupling plate 201 in such a way that along the clamping direction 205 the coupling rod 211 is fixed to the accommodation plate 132 and slidable with respect to the coupling plate 201.

The clamping arrangement 210 further comprises a driving system 220 for moving the clamping rod 211 along the clamping direction 205. The driving system 220 comprises a driving plate 221 to which the clamping rod 211 is non movably fixed at least along the clamping direction 205, wherein the driving plate 221 is movably with respect to the clamping plate 201. Hence, by moving the driving plate 221 in a longitudinal direction along the clamping direction 205, the clamping rods 211 fixed to the driving plate 221 are moved as well.

Furthermore, a spindle 222 is fixed to the coupling plate 201, wherein the driving plate 221 is movable coupled along the spindle 222. The driving system 220 comprises a spindle drive 223 mounted on the driving plate 221 for generating a driving force along the spindle 222. The spindle drive 223 comprises for example a rotatable threaded nut, which is rotated by a driving force. The spindle drive 223 rotates the threaded nut, such that a movement along the spindle 222, which is coupled to the threaded nut, is provided. The spindle 222 is further be guided through a through hole of the driving plate 221, such that a relative movement along the coupling direction 205 between the driving plate 221 and the spindle 222 is possible such that a movement of the driving plate 221 in the direction of the coupling direction 205 is possible. Hence, the clamping force is guided from the clamping rods 211 via the driving plate 221 to the coupling plate 201.

Furthermore, the accommodation plate 132 comprises a clamping groove 206 for accommodating the clamping rod 211 in such a manner that the clamping rod 211 is slidable along the clamping groove 206 and is movably fixed with the accommodation plate 132 along the clamping direction 205. The clamping groove 202 comprises specifically a lateral opening along a side edge of the accommodation plate 132. Furthermore, the clamping groove 206 is formed in such a manner, that an undercut (in a cross-sectional view) is formed, such as a T-shape. Hence, the clamping groove 206 comprises a T-shaped cross section and the clamping rod 211 comprises a T-shaped rod end 212 configured for fitting into the T-shaped clamping groove 206. The rod end 212 may form a single rod end or a plate-like rod end, to which a plurality of clamping rods 211 are mounted. The rod ends 212 form a plate that extends in horizontal direction h and may be inserted in the respectively shaped clamping groove 206. Hence, a more homogeneous clamping force in clamping direction 205 may be applied. Hence, the clamping rod 211 may slide from the side edge into the clamping groove 206, in particular along a horizontal direction h, that is perpendicular to the impact direction 103. If the clamping force is applied to the clamping rod 211, a form fit between the clamping rod 211 and the clamping groove 206 having an undercut is generated such that the accommodation plate 132 is pressed and drawn in the clamping direction 205 to the clamping plate 201 by the clamping rod 211.

A handling device may be provided to handle the accommodation plate 132, in particular to move the accommodation plate 132 to or away from the coupling plate 201 specifically along a horizontal direction h. Specifically, a sliding direction and hence the conveying direction is defined along the coupling protrusion 203 and the coupling groove 206, respectively. A sliding direction may be a horizontal direction h perpendicular to the impact direction 103. Hence, from one lateral side, the accommodation plate 132 may be slid over the coupling plate 201 for providing the material test and after the material test is accomplished, the accommodation plate 132 may move along the sliding direction away from the coupling plate 201.

FIG. 4 shows an embodiment of a specimen holder arrangement comprising a specimen holder 101. The accommodation plate 132 and the specimen holder 101 may form one modular exchangeable unit. The unit may be equipped with the specimen 102, beside and spaced apart from the apparatus 100. For example, the accommodation plate 132 may be fixed to the specimen holder 101. Furthermore, the accommodation plate 132 may comprise the accommodation grooves 133 and the specimen holder 101 comprises the accommodation protrusions 405 in order to detachably mount the specimen holder 101 to the accommodation plate 132.

The specimen 102 to be tested is arranged on a holder element 402 of the specimen holder, such as a clamping unit. Furthermore, the rod arrangement 110 is movable along the impact direction 103.

The arrangement 100 may also comprises an optical measuring device, in particular a high-speed camera, for optically measuring the specimen 102 under test, and/or a displacement sensor for measuring a displacement of a part of the specimen 102 under testing conditions, in particular under a treatment of the specimen 102 with the impact element 111.

The control device 408 is coupled to the respective sensors and the respective sensor data may be sent to a data acquisition unit, such as a central server unit, for processing the sensor data.

Furthermore, a handling device may be provided to handle the specimen holder 101, in particular to move the specimen holder 101 together with the accommodation plate 132 to or away from the coupling plate 201 specifically along a horizontal direction h. For example, the specimen holder 101 together with the accommodation plate 132 may be positioned on a trolley or conveyor of the handling device, wherein a handling platform of the trolley is on the same vertical height as the coupling plate 201, such that the specimen holder 101 together with the accommodation plate 132 can be pushed onto the coupling plate 201, wherein the coupling protrusion 203 slides along the coupling groove 202. The coupling plate 201 may comprise a stopper which terminates the sliding movement and thereby defines a predefined position.

The control unit 408 is further configured for controlling the handling device and the actuator 120 for equipping the holder accommodation section 131 with the specimen holder 102 with the specimen holder 101 and for conducting a material test. The control unit 408 may be coupled to the rod arrangement 110, the actuator 120, and the specimen holder 101 for transmitting control signals in order to control the apparatus 100 such that a self-acting activation of the apparatus 100 and a material test, respectively, can be provided. Additionally, the control unit 408 is coupled to the handling device and the driving system 220 in order to automatically clamp and release the accommodation plate 132.

Furthermore, control unit 408 may comprise specimen data including for example design and material parameters of the specimen 102 as well as predefined test procedures, such as information about the pressing force, the frequency of the rod arrangement 110 during a dynamic test, for conducting a respective material test. Hence, by the control unit 408 an automatic loading and unloading of the apparatus 200 can be provided as well as an automatic operation of the material test apparatus is provided.

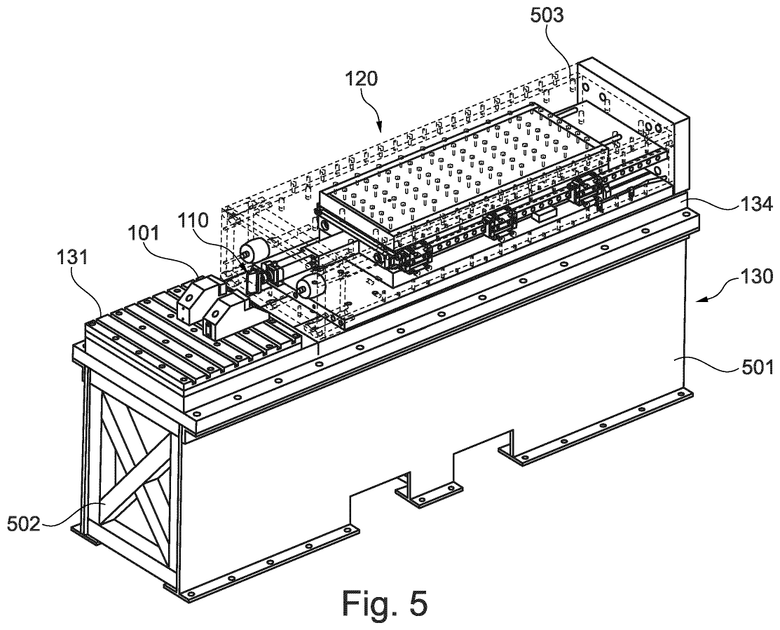

FIG. 5 shows an arrangement specifically with its supporting base 130 according to an exemplary embodiment of the present disclosure. The supporting base 130 is configured for providing a stiffness of more than 600 kN/mm. Hence, weight forces and dynamic forces may be transferred to the ground without causing vibrations which could negatively affect the testing procedure. The supporting base 130 is formed robustly for example by a framework 502 of steel rods which can be arranged on the ground. The stiffness may be provided by the robust above-described framework 502 of steel beams and additionally by a vertical shear panel 501.

The vertical shear panel 501 extends between the ground on the one side and the specimen holder 101, the rod arrangement 110 and the electromechanical actuator 130 on the other side (and for example between the supporting plate 134 and the ground). The shear panel 501 is in particular a vertical orientated sheet, in particular a metal sheet. By providing the vertical shear panel 501, forces (specifically shear forces) extending along the vertical direction v and also along the impact direction 103 are absorbed and damped by the vertical shear panel 501. The shear panel 501 is configured for having eigenfrequencies along the impact direction 103 of more than 300 Hz.

The supporting plate 134 forms a robust and stiff accommodation surface and may be arranged on the rigid framework 501 of steel beams of the supporting base 130. By arranging the supporting plate 134 within a horizontal plane, forces directed along the horizontal direction h are damped and absorbed efficiently.

The arrangement 100 further comprises a housing 503 for housing the actuator 120 and at least a part of the rod arrangement 110.

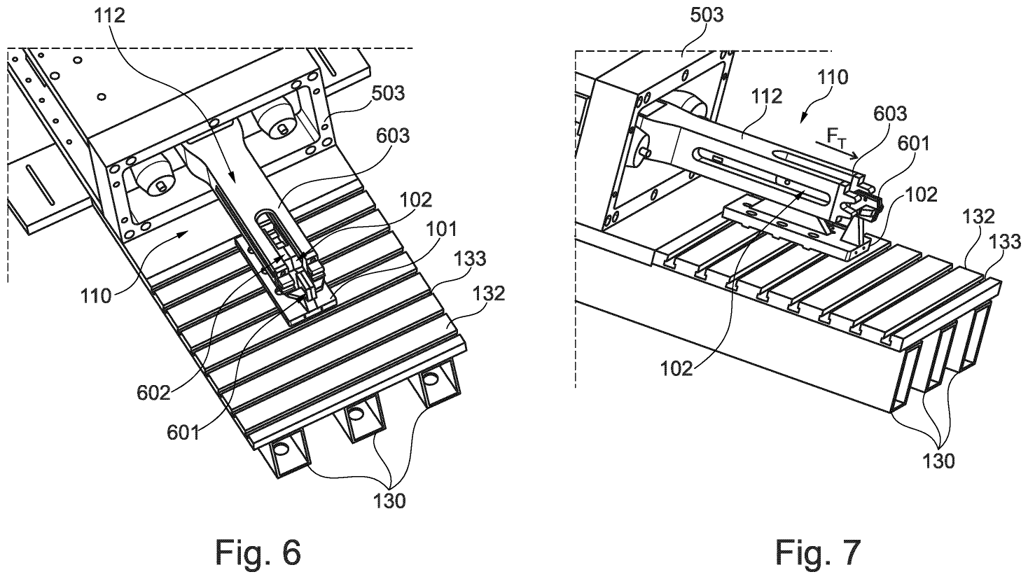

FIG. 6 and FIG. 7 show an exemplary embodiment of an apparatus 100 providing a rod arrangement 110 for applying a tractive force FT to the specimen 102. The specimen holder 101 is fixed to the accommodation plate 132. Furthermore, the specimen holder 101 comprises a clamping element 602, for example clamping jaws, for clamping the specimen 101 non-movably with respect to the supporting base 130. Spaced apart from the clamping element 602, a gripping element 601 is provided at the movable force transmitting rod 112. For example, the gripping element 601 is mounted at the free end of the force transmitting rod 112. Specifically, the force transmitting rod 112 comprises a split section and forms a fork like end section. The fork like end section 603 passes the specimen 102. In the end of the fork like end section 603, the gripping element 601 is arranged. The gripping element 601 grips the specimen 102 spaced apart with respect to the clamping section of the clamping element 602. The gripping element 601 may fix the specimen 102 for example by clamping or by a form fit fixation. Hence, if the force transmitting rod 112 is moved out of the housing 503 and hence along a respective horizontal moving direction, the gripping element 601 is moved away from the clamping element 602, such that a tractive force FT is transmitted to the specimen 102 to be tested.

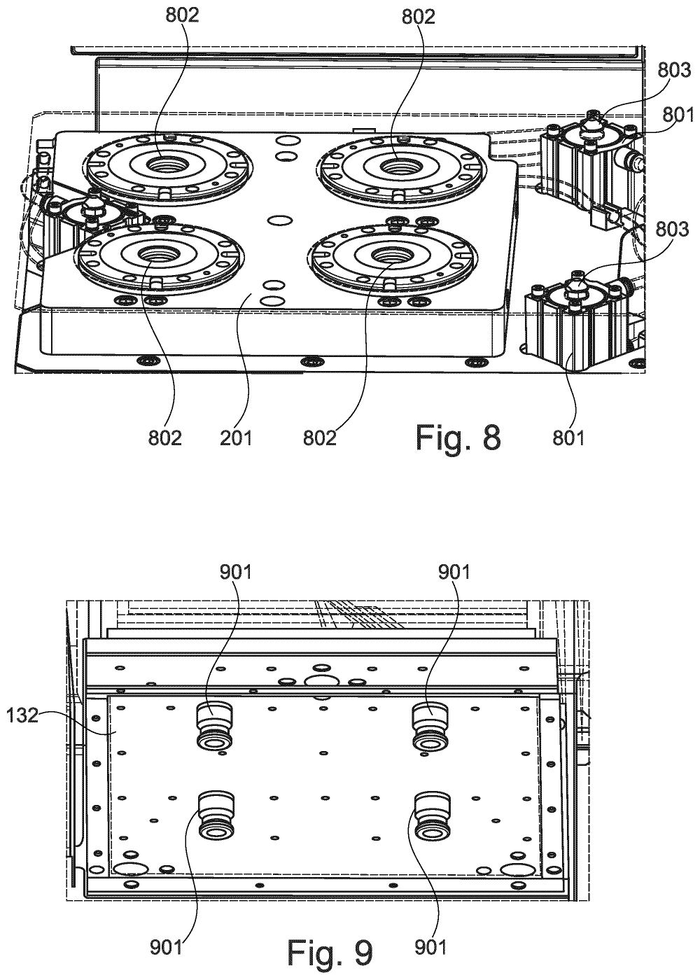

FIG. 8 shows a schematic view of a coupling plate 201 comprising accommodation holes 802 according to an exemplary embodiment. Respectively, FIG. 9 shows a schematic view of holder accommodation plate 132 of the holder accommodation section with mounting pins 801 according to an exemplary embodiment.

The lifting mechanism 801 comprises three lifting pistons 803 configured for being extendable (and retractable) between coupling plate 201 and the holder accommodation plate 132 for lifting and lowering the holder accommodation plate 132. The lifting pistons 803 may be driven by pneumatic, hydraulic or electric actuators.

The coupling plate 201 comprises accommodation holes 802 for receiving mounting pins 901 of the holder accommodation plate 132, wherein the holder accommodation plate 132 comprises controllable clamping means for fixing the mounting pins 901. Hence, the holder accommodation plate 132 may be moved along a vertical direction onto the coupling plate 201 for engaging the mounting pins 901 in the accommodation holes 802. In the accommodation hole 802, a controllable fixing mechanism may be installed. For example, the fixing mechanism may comprise respective clamping means which may be moved in a clamping position, if the mounting pin 901 is arranged within the accommodation hole 802.

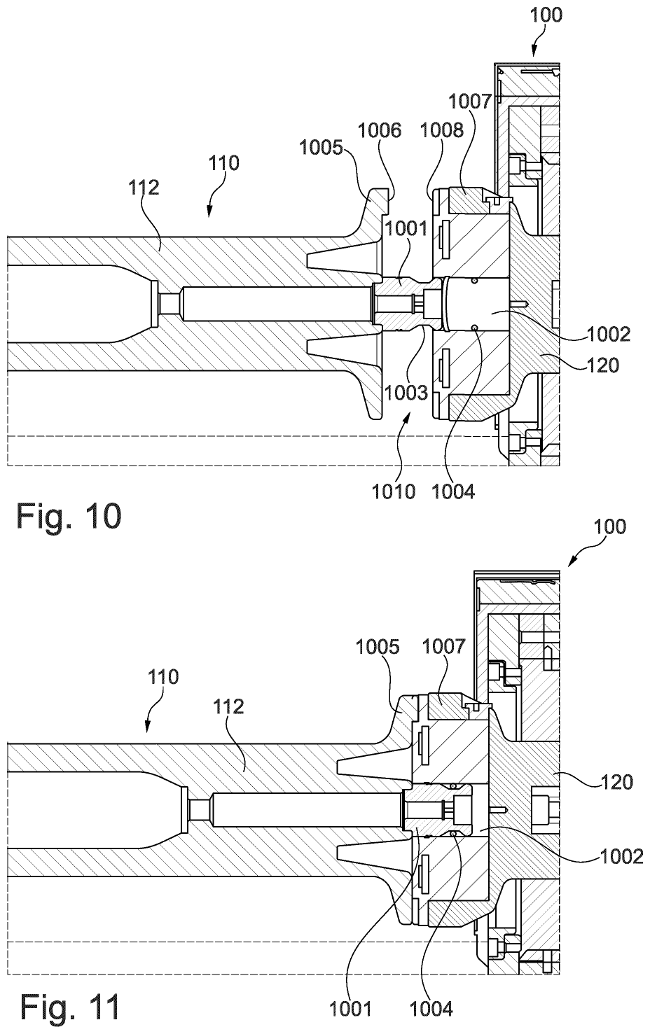

FIG. 10 and FIG. 11 show schematic views of a coupling device 1010 schematic according to an exemplary embodiment. FIG. 10 shows a position of the force transmitting rod of the rod arrangement 110 before coupling and FIG. 11 shows a position of the force transmitting rod of the rod arrangement 104 in coupling position.

In the shown embodiment, the coupling device 1010 comprises a coupling pin 1001 configured for being insertable in a receiving hole 1002 of a coupling section 1009 of the apparatus 100, wherein the coupling pin 1001 comprises a receiving section, in particular a groove 1003, for receiving a clamping element 1004. In the exemplary embodiment, the clamping element 1004 is formed by radially movable balls, in particular steel balls, of the apparatus 100.

The coupling pin 1001 extends in particular in a moving direction of the force transmitting rod. The balls as clamping elements 1004 are pretensioned in a radial direction (hence to the center axis of the force transmitting rod 112). Hence, upon movement of the coupling pin 1001 to the actuator 100, the balls in the receiving hole 1002 of the coupling section 1009 of the actuator 100 are pushed radially outwards until the groove 1003 of the clamping pin 1001 reaches the balls. In this position, the balls are pushed by the pretension force into the groove 1003, such that further relative axial movement (along the center axis of the force transmitting rod) is prevented.

The clamping elements 1004, e.g. the balls, may be pretensioned by respective springs.

Furthermore, the rod arrangement 1004 comprises a rod flange 1005 with abutting surfaces 1006 and the apparatus coupling section 1009 comprises an actuator flange 1007 with abutting surfaces 1008. As can be seen in FIG. 11, the abutting surfaces contact each other in a coupled position in FIG. 11 so that a large contact area can be formed for stabilizing the coupling against forces in radial direction.

Furthermore, the coupling device 1010 may also form a magnetic coupling. For example, the force transmitting rod 112 may comprise magnetic elements and the actuator 120 comprises at the coupling section 1009 a controllable electromagnetic device for selectively coupling the force transmitting rod to the actuator 120. Furthermore, the coupling device 1010 may also form a screw connection between the force transmitting rod 112 and the actuator 120. The actuator 120 may comprise a respective coupling mechanism, for detachably coupling the coupling device 108.

It should be noted that the term “comprising” does not exclude other elements or steps and the article “a” or “an” does not exclude a plurality. Also, elements described in association with different embodiments may be combined.

List of reference signs:

| | |
|---|---|
| 100 | apparatus |
| 101 | specimen holder |
| 102 | specimen |
| 103 | impact direction |
| 110 | rod arrangement |
| 111 | impact element |
| 112 | force transmitting rod |
| 113 | force sensor |
| 120 | actuator |
| 121 | slide |
| 122 | stator |
| 123 | stator table |
| 124 | further stator table |
| 130 | supporting base |
| 131 | holder accommodation section |
| 132 | accommodation plate |
| 133 | accommodation groove |
| 134 | supporting plate |
| 201 | coupling plate |
| 202 | coupling groove |
| 203 | coupling protrusion |
| 204 | tapered wall |
| 205 | clamping direction |
| 206 | clamping groove |
| 210 | clamping arrangement |
| 211 | clamping rod |
| 212 | rod end |
| 220 | driving system |
| 221 | driving plate |
| 222 | spindle |
| 223 | spindle drive |
| 402 | holder element |
| 405 | accommodation protrusion |
| 408 | control device |
| 501 | shear panel |
| 502 | support framework |
| 503 | housing |
| 601 | gripping element |
| 602 | clamping element |
| 603 | end section |
| 801 | lifting mechanism |
| 802 | accommodation hole |
| 803 | lifting piston |
| 901 | mounting pin |
| 1001 | coupling pin |
| 1002 | receiving hole |
| 1003 | groove |
| 1004 | clamping element |
| 1005 | rod flange |
| 1006 | abutting surfaces |
| 1007 | actuator flange |
| 1008 | abutting surfaces |
| 1009 | apparatus coupling section |
| 1010 | coupling device |
| v | vertical direction |
| h | horizontal direction |
| FT | tractive force |

1. An apparatus for material testing of a specimen, the apparatus comprising:
a specimen holder arrangement comprising a specimen holder for holding a specimen to be tested,
a rod arrangement for moving in a direction toward the specimen holder for transmitting a mechanical load to the specimen,
an actuator for moving at least one of the rod arrangement and the specimen holder with respect to each other along a horizontal impact direction, and
a supporting base, onto which the specimen holder arrangement, the rod arrangement and the actuator are mounted,
wherein the specimen holder arrangement is detachably coupled to a holder accommodation section of the supporting base.

2. The apparatus according to claim 1,
wherein the actuator and the rod arrangement are configured for conducting a static test for providing a pressing force to the specimen to be tested and/or for conducting a dynamic test for varying an impact force in a predefined time span.

3. The apparatus according to claim 1,
wherein the holder accommodation section comprises a holder accommodation plate onto which the specimen holder arrangement is detachably coupled.

4. The apparatus according to claim 3, comprising at least one of the following features:
wherein the holder accommodation plate comprises at least one mounting pin being engageable in a respective accommodation hole of a coupling plate of the accommodation section,
wherein a coupling plate of the accommodation section comprises at least one mounting pin being engageable in a respective accommodation hole of the holder accommodation plate,
wherein the accommodation section comprises controllable clamping means for fixing the mounting pins into the respective accommodation hole of the coupling plate,
wherein the apparatus comprises:
a lifting mechanism coupled to the coupling plate,
wherein the lifting mechanism is configured for lifting the holder accommodation plate from the coupling plate to thereby decoupling the holder accommodation plate from the coupling plate.

5.-6. (canceled)

7. The apparatus according to claim 4,
wherein the lifting mechanism comprises at least two lifting pistons configured for being extendable between the accommodation plate and the coupling plate for lifting and lowering the holder accommodation plate from the coupling plate.

8. The apparatus according to claim 3,
wherein the holder accommodation plate comprises at least one accommodation groove,
wherein the specimen holder arrangement comprises at least one accommodation protrusion,
wherein the accommodation protrusion is configured for being slidable within the accommodation groove for detachably coupling the specimen holder arrangement to the holder accommodation section,
wherein the accommodation groove is perpendicular to the impact direction.

9. The apparatus according to claim 3, comprising at least one of the following features:
wherein the holder accommodation section comprises a coupling plate configured for detachably coupling the accommodation plate
wherein the accommodation plate comprises at least one coupling groove and the coupling plate comprises a coupling protrusion,
wherein the coupling protrusion and the coupling groove are formed in such a manner that the coupling protrusion is slidable within the coupling groove,
wherein the coupling groove extends perpendicular to the impact direction.

10.-11. (canceled)

12. The apparatus according to claim 9,
wherein the coupling groove comprises tapered sidewalls forming a trapezoidal cross-section.

13. The apparatus according to claim 9,
wherein the coupling protrusion is a longitudinal ridge with tapered sidewalls forming a trapezoidal cross-section.

14. The apparatus according to claim 7,
wherein the accommodation section comprises a clamping arrangement for moving the accommodation plate to the coupling plate along a clamping direction such that accommodation plate is clampable to the coupling plate,
wherein the clamping direction is a vertical clamping direction.

15. The apparatus according to claim 14, comprising at least one of the following features:
wherein the clamping arrangement comprises at least one clamping rod configured for pulling the accommodation plate to the coupling plate along the clamping direction,
wherein the clamping rod is coupled to the accommodation plate and to the coupling plate in such a way that along the clamping direction the coupling rod is fixed to the accommodation plate and slidable with respect to the coupling plate,
wherein the clamping arrangement comprises a driving system for moving the clamping rod along the clamping direction,
wherein the driving system comprises a driving plate to which the clamping rod is non-movably fixed at least along the clamping direction,
wherein the driving plate is movably with respect to the clamping plate,
wherein a spindle is fixed to the coupling plate,
wherein the driving plate is movable coupled along the spindle,
wherein the driving system comprises a spindle drive mounted on the driving plate for generating a driving force along the spindle.

16.-19. (canceled)

20. The apparatus according to claim 15,
wherein the accommodation plate comprises a clamping groove for accommodating the clamping rod in such a manner that the clamping rod is slidable along the clamping groove and is movably fixed with the accommodation plate along the clamping direction.

21. The apparatus according to claim 20,
wherein the clamping groove comprises a T-shaped cross section and the clamping rod comprises a T-shaped rod end configured for fitting into the T-shaped clamping groove.

22. The apparatus according to claim 14,
wherein the supporting base comprises a supporting plate to which at least the clamping arrangement is mounted.

23. The apparatus according to claim 1,
wherein the supporting base comprises at least one vertical shear panel extending between the ground on the one side and the specimen holder arrangement, the rod arrangement and the actuator on the other side,
wherein the shear panel is a vertical orientated sheet.

24. The apparatus according to claim 15, further comprising at least one of the following:
a handling device,
wherein the handling device is configured to handle the accommodation plate, to move the accommodation plate to or away from the coupling plate,
a control unit for controlling the handling device and the actuator for equipping the holder accommodation section with the specimen holder and for conducting a material test,
wherein the rod arrangement further comprises a force sensor for measuring the impact force between the rod arrangement and the specimen to be tested,
an optical measuring device for optically measuring the specimen under test, and
a displacement sensor for measuring a displacement of a part of the specimen under testing conditions with the rod arrangement.

25.-27. (canceled)

28. The apparatus according claim 1,
wherein the rod arrangement comprises a coupling device arranged at an end of the force transmitting rod,
wherein the coupling device is detachably coupleable to the actuator of the apparatus.

29. The apparatus according claim 28,
wherein the coupling device comprises a coupling pin configured for being insertable in an actuator receiving hole of a coupling section of the apparatus,
wherein the coupling pin comprises a receiving section for receiving a clamping element with radially movable balls of the apparatus.

30. The apparatus according to claim 1,
wherein the specimen to be tested is a battery device.

31. A method for material testing of a specimen by an apparatus, the method comprising:
coupling a specimen holder arrangement detachably to a holder accommodation section of the supporting base,
moving at least one of a rod arrangement and the specimen holder arrangement with respect to each other for transmitting a mechanical load to a specimen for material testing the specimen.
